(19) United States
(12) Patent Application Publication
(10) Pub. No.: US 2026/0224149 A1
(43) Pub. Date: Aug. 6, 2026

(54) BIOLOGICAL INFORMATION MEASUREMENT DEVICE, INFORMATION PROCESSING SYSTEM, AND STORAGE MEDIUM

(71) Applicant: OMRON HEALTHCARE CO., LTD., Muko-shi, (JP)

(72) Inventors: Kentaro MORI, Kyoto (JP); Tsuyoshi KITAGAWA, Kyoto (JP)

(73) Assignee: OMRON HEALTHCARE CO., LTD., Muko-shi, (JP)

(21) Appl. No.: 19/445,820

(22) Filed: Jan. 12, 2026

(30) Foreign Application Priority Data
Feb. 5, 2025 (JP) 2025-017922

(51) Int. Cl. A61B 5/339; A61B 5/00; A61B 5/0205; A61B 5/022; A61B 5/349; G16H 40/67

(52) U.S. Cl. CPC A61B 5/339; A61B 5/0205; A61B 5/349; A61B 5/746; G16H 40/67; A61B 5/022

(57) ABSTRACT

A biological information device measures blood pressure and detects arrhythmia. It features a blood pressure unit, an arrhythmia detector, a display, storage, a communication unit, and a control unit. Normally, it displays the blood pressure reading. However, if arrhythmia is detected during measurement, instead of displaying the blood pressure, the device shows an arrhythmia alert. The device stores the blood pressure value regardless of arrhythmia detection. Only after receiving explicit permission (a permission signal) following the arrhythmia alert, the stored blood pressure value will be shown on the display. This ensures users are aware of potential inaccuracies due to arrhythmia.

### CROSS-REFERENCE TO RELATED APPLICATIONS

The disclosure of Japanese Patent Application No. JP 2025-017922, filed on Feb. 5, 2025 including the specification, drawings and abstract is incorporated herein by reference in its entirety.

### TECHNICAL FIELD

The present invention relates to a biological information measurement device capable of measuring blood pressure of a living body, an information processing system, and a program.

### BACKGROUND ART

In recent years, it has become widespread to perform health management by measuring information regarding the body and health of an individual such as a blood pressure value with a measuring instrument and recording and analyzing the measurement result. In particular, since arrhythmia such as atrial fibrillation (AF) may lead to a brain/cardiovascular disease, it is desirable that the user himself/herself can easily recognize such an abnormality.

In this regard, for example, there has been proposed a sphygmomanometer that displays, in a case where atrial fibrillation has been detected at the time of blood pressure measurement, the fact that there has been atrial fibrillation together with a blood pressure measurement result (for example, Patent Document 1). Furthermore, in a system for assisting diagnosis by a doctor, it has been proposed to display, in a case where arrhythmia has been detected at the time of measurement, a graph together with measurement values (blood pressure, pulse, etc.) of daily biological information on a patient, the graph displaying a mark indicating the fact that arrhythmia has been detected (for example, Patent Document 2).

According to the sphygmomanometer as described in Patent Document 1, the user can quickly recognize the presence of arrhythmia while performing daily health management of blood pressure measurement. Furthermore, according to the medical care assistance system as described in Patent Document 2, the medical professionals can grasp the presence or absence of arrhythmia in addition to the measurement data such as the daily blood pressure value of the patient.

### PRIOR ART DOCUMENTS

### Patent Documents

Patent Document 1: JP 2024-039189 A
Patent Document 2: JP 2023-151323 A

### SUMMARY

However, in a device used by a patient himself/herself (a user who is not a medical professional), if the blood pressure value is displayed as the measurement result even in a case where arrhythmia occurs during the blood pressure measurement, the patient may focus on the displayed blood pressure value and overlook the occurrence of the arrhythmia. In such case, the patient erroneously recognizes the inaccurate blood pressure value of the time when arrhythmia has been detected as the blood pressure value as a result of appropriate measurement.

On the other hand, even if the blood pressure value is inaccurate, the fact that arrhythmia has been detected and the measurement value at that time are useful information for the doctor. Also for the patient, if the blood pressure value of the time when arrhythmia has been detected is confirmed after understanding that the measurement value is inaccurate, that blood pressure value is useful as reference information.

In view of the above circumstances, an object of the present invention is to provide a technology capable of preventing an inaccurate measurement value from being indicated to a user as a blood pressure measurement result and of confirming, as required, a measurement value of the time when arrhythmia has been detected during the blood pressure measurement.

In order to solve the above problem, the present invention adopts the following configuration. The configuration is a biological information measurement device including:
a blood pressure measurement unit that acquires information for measuring blood pressure of a living body;
an arrhythmia information acquisition unit that acquires information related to presence or absence of arrhythmia in the living body during measurement of the blood pressure;
a display unit including an area capable of displaying at least a blood pressure value measured;
a storage unit that stores at least the blood pressure value;
a communication unit for communicating with another device; and
a control unit, wherein
the control unit
   calculates the blood pressure on the basis of the information acquired by the blood pressure measurement unit,
   causes, in a case where the information acquired by the arrhythmia information acquisition unit indicates that arrhythmia during the measurement of the blood pressure has been present, the display unit not to display a value of the blood pressure measured but to display information of a fact that arrhythmia has been present, and
   causes, after causing the information of the fact that arrhythmia has been present to be displayed, in a case where a permission signal to permit display of a blood pressure value measured when the arrhythmia has been present is received via the communication unit, the display unit to display the blood pressure value.

Note that the information related to the presence or absence of arrhythmia acquired by the arrhythmia information acquisition unit may be information on the presence or absence of arrhythmia detected by another biological information measurement device or a biological signal itself measured by another device. Furthermore, “case where a permission signal is received” means not only the timing at which the permission signal is received but also after the reception. Examples of “arrhythmia” include atrial fibrillation, atrial flutter, and premature contraction.

According to such a configuration, in a case where arrhythmia has been present during the blood pressure measurement, only the fact that arrhythmia has been present is displayed without displaying the blood pressure value. Therefore, it is possible to prevent the inaccurate blood pressure measurement result from being indicated to the user and, in a case where the signal to permit the display of the measurement value of the time when the arrhythmia has been detected is received, it is possible to refer to the measurement value.

The biological information measurement device may further include a storage unit that holds information related to measurement of the blood pressure performed in a past, the information including information related to a date and time of the measurement and presence or absence of arrhythmia during the measurement at the date and time, and
the control unit
   may receive, via an input unit, a request to display, on the display unit, the information related to the measurement of the blood pressure performed in the past, the information being held in the storage unit, and
   may cause, in a case where the information related to the measurement of the blood pressure related to the request to display includes information of the fact that arrhythmia has been present during the measurement, the display unit not to display a value of the blood pressure at the time of the measurement performed in the past but to display the information of the fact that arrhythmia has been present.

According to such a configuration, the user can view the past measurement history, and even in a case where arrhythmia has been detected at the time of the past measurement, it is possible to prevent an inaccurate blood pressure measurement result from being indicated to the user, and to notify the user of the arrhythmia detected during the blood pressure measurement.

Furthermore, the arrhythmia information acquisition unit may acquire the information related to presence or absence of arrhythmia in the living body on the basis of pulse wave information acquired by the blood pressure measurement unit. For example, in a case where the blood pressure measurement unit calculates the blood pressure on the basis of the pressure pulse wave, arrhythmia can be detected on the basis of the pressure pulse wave.

Furthermore, the biological information measurement device may further include an electrocardiographic waveform acquisition unit that acquires an electrocardiographic waveform of the living body, wherein the arrhythmia information acquisition unit may acquire the information related to presence or absence of arrhythmia in the living body on the basis of the electrocardiographic waveform during the measurement of the blood pressure.

Furthermore, the present invention can also be regarded as an information processing system as follows. The information processing system is
an information processing system including: the biological information measurement device as described above; and an information processing terminal communicatively connected to the biological information measurement device, wherein
the biological information measurement device
   transmits, to the information processing terminal, information at least related to presence or absence of arrhythmia during the measurement of the blood pressure, and wherein
the information processing terminal includes:
   a terminal-side display unit that displays, in a case where the information of the fact that arrhythmia has been present during the measurement of the blood pressure is received, at least the fact; and
   an input unit that receives a display permission operation that is an operation for permitting display of the blood pressure value measured when the arrhythmia has been present, and
   transmits, in a case where the display permission operation is received, the permission signal to the biological information measurement device.

Here, the “information processing terminal” communicatively connected to the biological information measurement device can be, for example, a terminal used by a doctor, a terminal owned by a user (such as a smartphone), a server device that provides software as a service (SAAS), or the like.

Furthermore, the biological information measurement device may transmit information on the blood pressure value measured to the information processing terminal, and
the information processing terminal may display the information on the blood pressure value received on the terminal-side display unit, and may display, in a case where the blood pressure value to be displayed is a blood pressure value measured when the arrhythmia has been detected, a fact that the blood pressure value displayed is a blood pressure value measured when arrhythmia has been detected together with the blood pressure value.

According to such a configuration, for example, the doctor refers to the past measurement data at the time of medical examination, explains to the patient the inaccuracy of the measurement value at the time when arrhythmia has occurred, and then permits the display of the measurement value on the biological information measurement device, so that it is possible to prevent erroneous recognition by the user, and the doctor can also use the measurement data of the time when arrhythmia has occurred for medical care.

Furthermore, the biological information measurement device may transmit the information on the blood pressure value measured to the information processing terminal, and
the information processing terminal may suspend, in a case where the blood pressure value received is a blood pressure value measured when arrhythmia has been detected, display of the blood pressure value on the terminal-side display unit and display a fact that the blood pressure value received is the blood pressure value of a case where arrhythmia has been detected during the measurement on the terminal-side display unit, and may display, only in a case where the display permission operation is received, the blood pressure value received on the terminal-side display unit.

According to such a configuration, for example, in a case where the information processing terminal is a terminal used by the user, the user himself/herself confirms that the measurement value is a value of a case where arrhythmia has been detected during the measurement (and that the calculated blood pressure value is accordingly inaccurate), and performs an operation of approving the confirmation on the terminal, so that the measurement value can be confirmed in both the information processing terminal and the biological information measurement device.

Furthermore, the present invention can also be regarded as an information processing system as follows. The information processing system is
an information processing system including: a biological information measurement device configured to be able to measure at least blood pressure of a living body; and an information processing terminal communicatively connected to the biological information measurement device, wherein
the biological information measurement device
   acquires information for measuring the blood pressure of the living body and calculates a blood pressure value,
   acquires determination information for determining presence or absence of arrhythmia in the living body during measurement of the blood pressure and transmits the determination information to the information processing terminal,
   suspends, in a case where a signal of a fact that arrhythmia during the measurement of the blood pressure has been present is received from the information processing terminal, display of a value of the blood pressure measured and displays information of the fact that arrhythmia has been present, and
   displays, after displaying the information of the fact that arrhythmia has been present, in a case where a permission signal to permit display of a blood pressure value measured when the arrhythmia has been present is received from the information processing terminal, the blood pressure value, and wherein
the information processing terminal
   receives the determination information from the biological information measurement device,
   determines presence or absence of arrhythmia during measurement of the blood pressure of the living body on the basis of the determination information,
   transmits, in a case where it is determined that arrhythmia has been present during the measurement of the blood pressure of the living body, information of a fact that arrhythmia has been present during the measurement of the blood pressure of the living body to the biological information measurement device and receives a display permission operation that is an operation for permitting display of a blood pressure value measured when the arrhythmia has been present, and
   transmits, in a case where the display permission operation is received, the permission signal to the biological information measurement device.

According to such a system, it is not necessary to determine the presence or absence of arrhythmia during the blood pressure measurement by the biological information measurement device, and the calculation load of the biological information measurement device can be reduced. As a result, even if the biological information measurement device does not include a high-performance calculation device, it is possible to prevent an inaccurate blood pressure measurement result from being indicated to the user and to notify the user of the arrhythmia detected during the blood pressure measurement.

Furthermore, the biological information measurement device may include an electrocardiographic waveform acquisition unit that acquires an electrocardiographic waveform of the living body, and the determination information may be the electrocardiographic waveform.

Furthermore, the present invention can also be regarded as a program for causing the information processing device to function as the above-described information processing terminals, and a computer-readable recording medium in which such a program is non-transiently recorded.

Note that each of configurations and processing described above can be combined with each other to constitute the present invention as long as no technical contradiction occurs.

According to the present invention, it is possible to prevent an inaccurate blood pressure measurement result from being indicated to a user and to confirm, as required, a measurement value of the time when the arrhythmia has been detected during the blood pressure measurement.

### BRIEF DESCRIPTION OF THE DRAWINGS

FIG. 1 is a schematic diagram schematically illustrating an information processing system according to a first embodiment;

FIG. 2 is a block diagram schematically illustrating a device configuration of a biological information measurement device according to the first embodiment;

FIG. 3 is a block diagram illustrating functional modules implemented by a control unit of the biological information measurement device according to the first embodiment;

FIG. 4 is a flowchart illustrating a flow of blood pressure measurement processing by the biological information measurement device according to the first embodiment;

FIG. 5A is a first diagram illustrating an example of a measurement result display screen in the biological information measurement device when arrhythmia has been detected during blood pressure measurement;

FIG. 5B is a second diagram illustrating an example of the measurement result display screen in the biological information measurement device when arrhythmia has been detected during blood pressure measurement;

FIG. 6 is a flowchart illustrating a flow of processing when a history viewing mode is executed in a blood pressure measurement device according to the first embodiment;

FIG. 7 is a block diagram schematically illustrating a functional configuration of a doctor terminal according to the first embodiment;

FIG. 8A is a first diagram illustrating an example of a display screen in the doctor terminal when arrhythmia has been detected during blood pressure measurement;

FIG. 8B is a second diagram illustrating an example of the display screen in the doctor terminal when arrhythmia has been detected during blood pressure measurement;

FIG. 9 is a diagram illustrating a flow of processing related to transmission and reception of information between the biological information measurement device and the doctor terminal in the information processing system according to the first embodiment;

FIG. 10 is a block diagram schematically illustrating a device configuration of a biological information measurement device according to a modification of the first embodiment;

FIG. 11 is a block diagram illustrating functional modules implemented by a control unit of the biological information measurement device according to the modification of the first embodiment;

FIG. 12 is a block diagram schematically illustrating a device configuration of a biological information measurement device according to another modification of the first embodiment;

FIG. 13 is a block diagram illustrating functional modules implemented by a control unit of the biological information measurement device according to another modification of the first embodiment;

FIG. 14 is a schematic diagram schematically illustrating an information processing system according to a second embodiment;

FIG. 15 is a diagram illustrating a flow of processing related to transmission and reception of information among the biological information measurement device, a server device, and the doctor terminal in the information processing system according to the second embodiment;

FIG. 16 is a block diagram schematically illustrating an information processing system according to a third embodiment;

FIG. 17 is a block diagram illustrating functional modules implemented by a control unit of an information processing terminal of the third embodiment;

FIG. 18 is a first flowchart illustrating flows of processing of each of the biological information measurement device and the information processing terminal and transmission and reception of information in the information processing system according to the third embodiment;

FIG. 19 is a second flowchart illustrating flows of processing of each of the biological information measurement device and the information processing terminal and transmission and reception of information in the information processing system according to the third embodiment; and

FIG. 20 is a third flowchart illustrating flows of processing of each of the biological information measurement device and the information processing terminal and transmission and reception of information in the information processing system according to the third embodiment.

### DESCRIPTION OF THE EMBODIMENTS

### First Embodiment

Hereinafter, specific embodiments of the present invention will be described on the basis of the drawings. However, the specific detail of each configuration described in the embodiments described below, the arrangement relationship thereof, and the like are not intended to limit the scope of the present invention only to them unless otherwise specified.

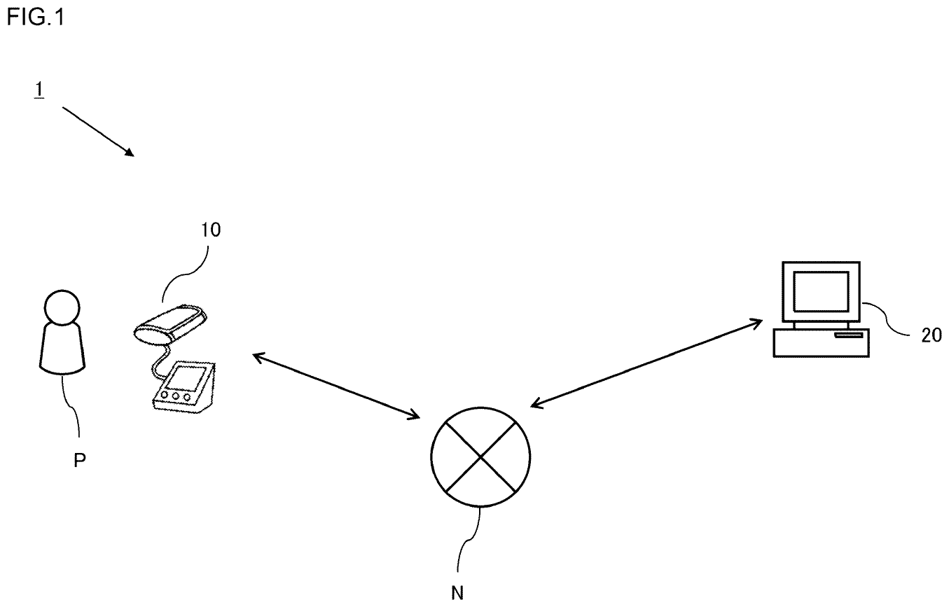

The present invention can be applied to, for example, an information processing system 1 as illustrated in FIG. 1 and a biological information measurement device 10 as a component of the information processing system. FIG. 1 is a schematic diagram illustrating a schematic configuration of the information processing system 1 according to the present embodiment. As illustrated in FIG. 1, the information processing system 1 includes a biological information measurement device 10 used by a patient P and a doctor terminal 20 used by a doctor, and these configurations can communicate with each other via a communication network N.

The information processing system 1 according to the present embodiment transmits a biological information measurement value such as a blood pressure value and a pulse rate measured by a user (patient) at home or the like to the doctor terminal 20 via the network N and provides a medical professional with the information, thereby assisting the doctor in providing the patient with treatment. Hereinafter, each configuration of the system will be described in detail.

### (Device Configuration of Biological Information Measurement Device)

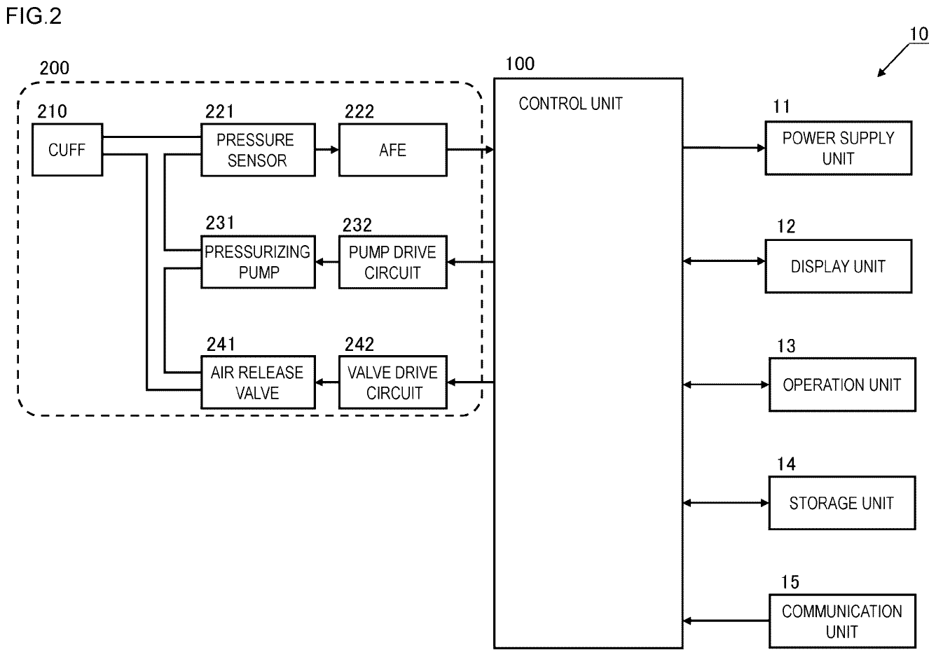

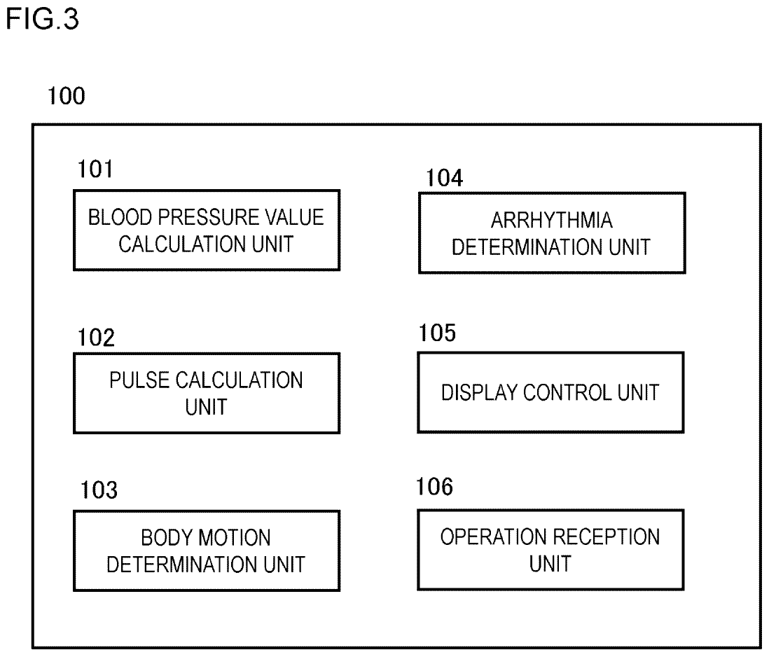

FIG. 2 is a block diagram schematically illustrating a device configuration of the biological information measurement device 10, and FIG. 3 is a block diagram illustrating an example of functional modules implemented by a control unit 100 of the biological information measurement device 10.

As illustrated in FIG. 1, the biological information measurement device 10 according to the present embodiment schematically includes components such as a power supply unit 11, a display unit 12, an operation unit 13, a storage unit 14, a communication unit 15, the control unit 100, and a blood pressure measurement unit 200, and has a configuration similar to that of a general portable upper-arm sphygmomanometer including a main body housing, a cuff, and an air tube as an appearance although not illustrated.

The power supply unit 11 is a power source of the biological information measurement device 10, and may be, for example, a rechargeable secondary battery such as a lithium ion battery, but is not limited thereto, and may be a primary battery, a terminal connectable to an external power supply, or the like.

The display unit 12 is a functional unit for displaying various types of information, and can be, for example, a display device such as a liquid crystal display (LCD). Furthermore, the operation unit 13 is a functional unit that receives a user operation such as turning on/off of a power supply, execution of measurement, and selection/decision of a menu, and can be configured by an operation switch and the like. Note that a touch panel display can also be adopted as a configuration that serves as both the display unit 12 and the operation unit 13.

The storage unit 14 includes a main storage device such as a random access memory (RAM) and a read only memory (ROM), and an auxiliary storage device such as a hard disk drive (HDD), a solid state device (SSD), and a removable medium, and stores various types of information such as an application program, measurement results such as a blood pressure value and a pulse, information on the presence or absence of arrhythmia to be described later, and other acquired biological information. Note that the measurement results of the blood pressure and the like and other acquired biological information may be stored in the storage unit 14 in association with time information such as the measurement (acquisition) time. As the time information, for example, time information clocked by referring to a real time clock (RTC) of the control unit 100 to be described later can be used. Furthermore, the auxiliary storage device may be configured to be detachable from the main body housing.

The communication unit 15 is a configuration for connecting to the communication network N, and can adopt an appropriate communication interface according to the communication network to be connected, such as a wired communication port or a wireless communication antenna.

The control unit 100 is a functional unit that controls the entire biological information measurement device 10, and includes, for example, a processor such as a central processing unit (CPU). When receiving a user's operation via the operation unit 13, the control unit 100 controls each component of the biological information measurement device 10 to execute various processing such as blood pressure measurement and presentation of various types of information according to a predetermined program. The predetermined program is stored in the storage unit 14 and read therefrom. As the processor, in addition to the CPU, a graphic processing unit (GPU), a field programmable grid array (FPGA), an application specific integrated circuit (ASIC), or the like can be adopted.

Furthermore, functional modules illustrated in FIG. 3, including a blood pressure value calculation unit 101, a pulse calculation unit 102, a body motion determination unit 103, an arrhythmia determination unit 104, a display control unit 105, and an operation reception unit 106 are implemented by the control unit 100. These functional modules will be described in detail later.

The blood pressure measurement unit 200 includes a cuff 210, a pressure sensor 221, an analog front end (AFE) 222 including an analog-to-digital (A/D) converter, a pressurizing pump 231, a pump drive circuit 232, an air release valve 241, and a valve drive circuit 242, and is a functional unit that measures the blood pressure of the user by a so-called oscillometric method. The blood pressure measurement by the oscillometric method is a well-known technology, and thus a detailed description thereof will be omitted.

The cuff 210 is a member used with being wound around the upper arm of the user and includes the pressurizing pump 231 (pump drive circuit 232) disposed in the main body housing, an air bladder (not illustrated) communicating with the air release valve 241 (valve drive circuit 242) via an air tube (not illustrated), a belt (not illustrated) incorporating the air bladder, the pressure sensor 221 provided on the belt, and the like.

Note that the belt of the cuff 210 is provided with a fixing unit (for example, a hook-and-loop fastener) for fixing the cuff 210 to the upper arm of the user, and the cuff 210 is wound around the upper arm of the user by the belt when blood pressure measurement is performed using the biological information measurement device 10.

For example, a piezoresistive sensor including a piezoelectric element can be adopted as the pressure sensor 221, and at least a pressure pulse wave of the user is acquired. Furthermore, the biological information measurement device 10 according to the present embodiment calculates the blood pressure and the pulse of the user on the basis of the pressure pulse wave acquired by the pressure sensor 221, and detects the presence or absence of the body motion and arrhythmia of the user during the blood pressure measurement as described later.

At the time of blood pressure measurement, the control unit 100 controls the pressurizing pump 231 (pump drive circuit 232) and the air release valve 241 (valve drive circuit 242) to adjust the cuff pressure of the cuff 210. Specifically, control is performed such that the pressurizing pump 231 is driven to send air to the cuff 210 to inflate the cuff 210 (increase the cuff pressure). In this way, after the blood flow is once inhibited by compressing the blood vessel of the upper arm of the user, control is performed such that the pressurizing pump 231 is stopped to open the air release valve 241 and the air is gradually released from the cuff 210 to contract the cuff 210 (reduce the cuff pressure).

Next, functional modules illustrated in FIG. 3 will be described. The blood pressure value calculation unit 101 calculates the blood pressure value of the user on the basis of the pressure pulse wave acquired by the pressure sensor 221. Note that, in the present embodiment, the blood pressure value is calculated by the oscillometric method, but it is also possible to provide a microphone in the cuff 210 and calculate the blood pressure value by, for example, a Korotkoff method of detecting a Korotkoff sound.

The pulse calculation unit 102 calculates the pulse rate of the user on the basis of the pressure pulse wave (such as the peak number of the pulse wave) acquired by the pressure sensor 221. The body motion determination unit 103 determines the presence or absence and the number of times of body motion during blood pressure measurement on the basis of the pressure pulse wave (such as disturbance of pulse waveform) acquired by the pressure sensor 221.

Furthermore, the arrhythmia determination unit 104 determines (detects) the presence or absence of arrhythmia during the blood pressure measurement on the basis of the pressure pulse wave (information on the time interval between the peaks of the pulse wave, and the like) acquired by the pressure sensor 221. That is, in the present embodiment, a combination of the arrhythmia determination unit 104 and the pressure sensor 221 corresponds to an arrhythmia information acquisition unit.

The display control unit 105 controls the display unit 12 (the display contents thereof). For example, the display control unit 105 causes the display unit 12 to display menu selection, measurement results, past history data, and the like, and specific display contents will be described later.

The operation reception unit 106 receives an operation input by a user via the operation unit 13. For example, when the user presses a measurement start button, an item selection button, a decision button, or the like (each including an interface expressed on an image), the biological information measurement device 10 is controlled to perform the associated operation.

### (Flow of Blood Pressure Measurement Processing)

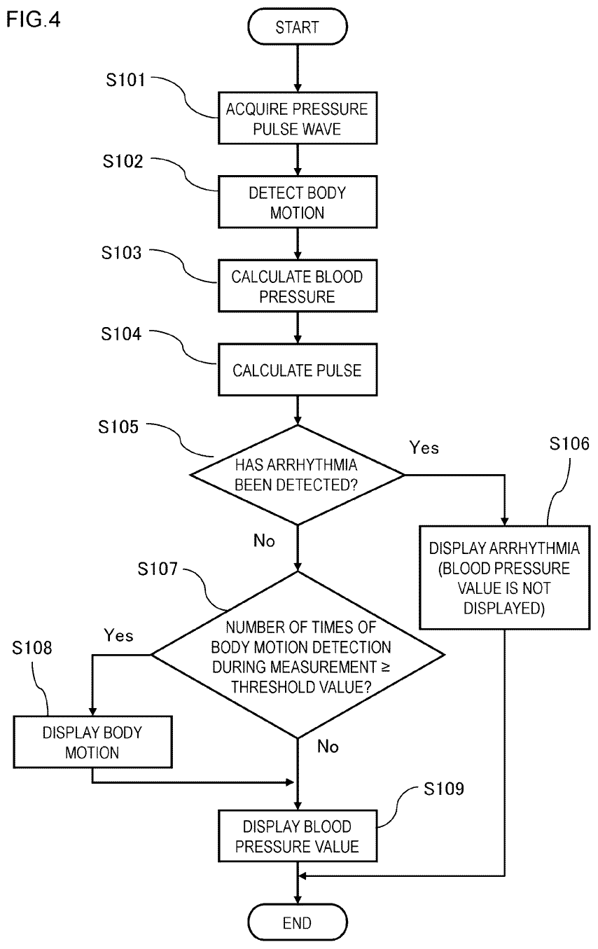

Subsequently, a flow of processing when measurement of blood pressure is executed by the biological information measurement device 10 will be described on the basis of FIG. 4. FIG. 4 is a flowchart illustrating a flow of processing by the biological information measurement device 10 when measurement of the blood pressure is performed in a normal mode. Prior to the execution of the blood pressure measurement, the user winds the cuff 210 around his/her upper arm, and then instructs execution of the blood pressure measurement in the normal mode via the operation unit 13.

After receiving the instruction of the blood pressure measurement, the biological information measurement device 10 acquires the pressure pulse wave of the user by the blood pressure measurement unit 200 (S101). Based on the acquired pressure pulse wave, the control unit 100 (the blood pressure value calculation unit 101, the pulse calculation unit 102, and the body motion determination unit 103) detects the body motion of the user (S102), calculates the blood pressure value (S103), and calculates the pulse rate (S104). In addition, the control unit 100 (arrhythmia determination unit 104) determines the presence or absence of arrhythmia during blood pressure measurement on the basis of the acquired pressure pulse wave (S105).

In a case where it is determined in step S105 that arrhythmia has been present during the blood pressure measurement, the control unit 100 (display control unit 105) does not display the calculated blood pressure value but displays, as a measurement result, the fact that arrhythmia has been present on the display unit 12 (S106).

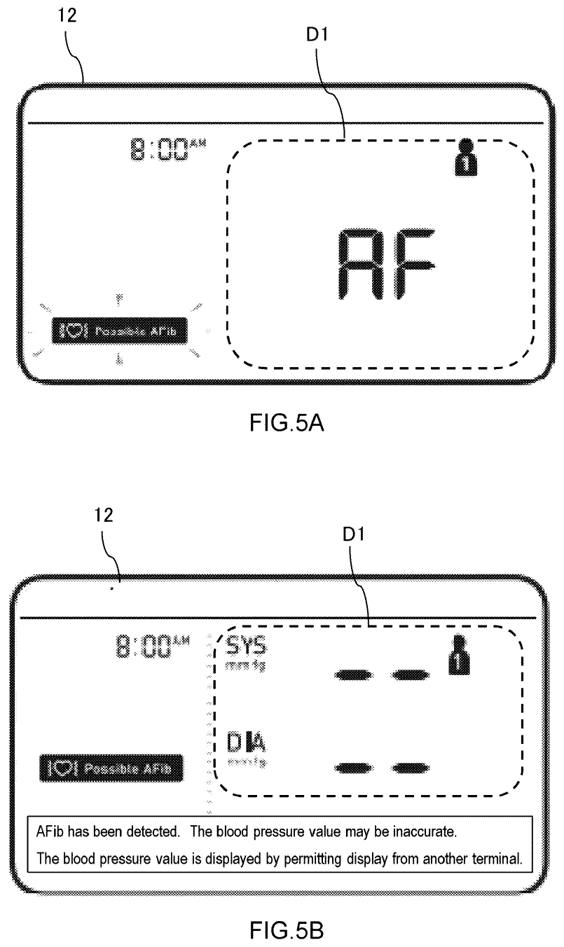

FIGS. 5A and 5B illustrate an example of the display unit 12 when displaying the measurement result of a case where arrhythmia has been detected during the blood pressure measurement in this manner. In the example illustrated in FIG. 5A, in a blood pressure display area D1 for displaying the measured blood pressure value of the display unit 12, the blood pressure value is not displayed, but “AF” indicating that arrhythmia (in this case, atrial fibrillation) has been detected is displayed. Furthermore, a mark indicating that atrial fibrillation has been detected is also displayed in a blinking manner on the lower left side of the screen.

Furthermore, in the screen display example illustrated in FIG. 5B, in the blood pressure display area D1 for displaying the measured blood pressure value, the blood pressure value is not displayed, and the display is left blank. On the other hand, in an area other than the blood pressure display area D1, display is provided to inform the fact that arrhythmia (AFib) has been detected and the fact that the calculated blood pressure value is inaccurate, and to provide guidance that the blood pressure value will be displayed on the display unit 12 when the operation of display permission is performed by another device. The operation of display permission will be described later.

On the other hand, in a case where it is determined in step S105 that arrhythmia has been absent during the blood pressure measurement, the control unit 100 (body motion determination unit 103) determines whether or not the number of times of body motion detected during the blood pressure measurement exceeds a predetermined threshold value (S107). In a case where it is determined in step S107 that the number of times of body motion is equal to or less than the threshold value, the control unit 100 (display control unit 105) displays the blood pressure value in the blood pressure display area D1 of the display unit 12 (S109).

In a case where it is determined in step S107 that the number of times of body motion exceeds the threshold value, the control unit 100 (display control unit 105) displays the fact that body motion has been detected (which may result in the low accuracy of the blood pressure value) in an area other than the blood pressure display area D1 of the display unit 12 (S108), and displays the blood pressure value in the blood pressure display area D1 (S109). Note that step S108 and step S109 may be executed simultaneously.

After displaying the blood pressure measurement result on the display unit 12 in step S106 or step S109, the biological information measurement device 10 stores the measurement data (blood pressure value, pulse rate, number of times of body motion, presence or absence of arrhythmia at the time of blood pressure measurement, and the like; the same applies hereinafter) in the storage unit 14 in association with the information on the measurement time, transmits the measurement data to the doctor terminal 20 via the communication unit 15, and ends the series of processing.

With the display of the measurement result as described above, in a case where arrhythmia has been detected during the blood pressure measurement, it is possible to prevent an inaccurate blood pressure measurement result from being indicated to the user and to notify the user of the arrhythmia detected during the blood pressure measurement. Furthermore, even in a case where arrhythmia has not been detected, when the number of times of body motion exceeds the threshold value, it is possible to alert the user by displaying that the accuracy of the calculated blood pressure value has decreased together with the blood pressure value.

### (Flow of Past Data Display Processing)

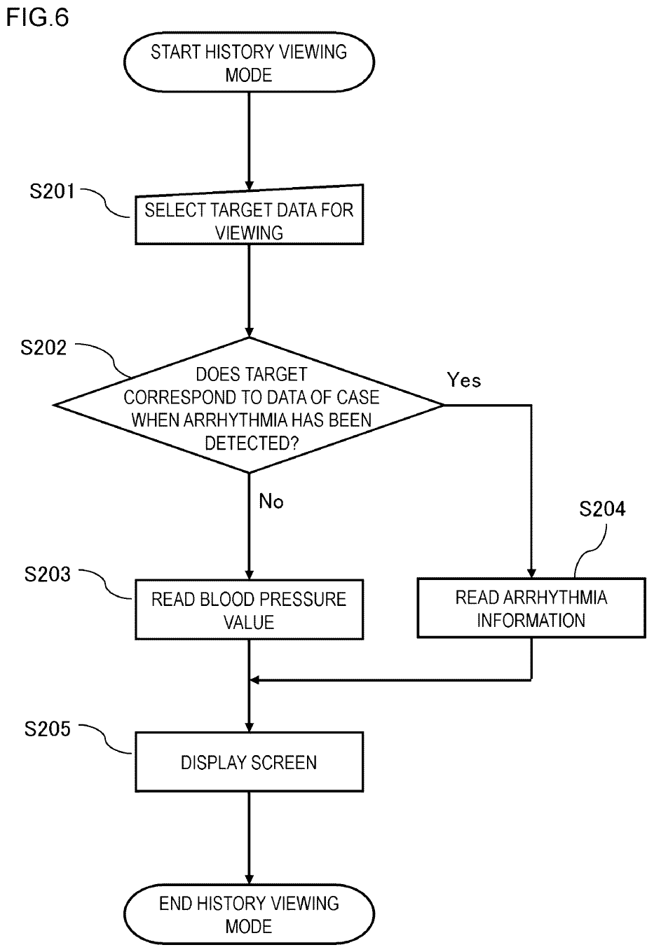

Subsequently, a flow of processing in the biological information measurement device 10 when the past measurement data stored in the storage unit 14 is displayed on the display unit 12 will be described on the basis of FIG. 6. FIG. 6 is a flowchart illustrating a flow of processing of referring to past measurement data (history viewing mode) in the biological information measurement device 10. As illustrated in FIG. 6, first, past measurement data (for example, which is read from the storage unit 14 and displayed as a list on the display unit 12) that the user wants to refer to is selected via the operation unit 13 (S201).

Next, the control unit 100 determines whether or not the measurement data according to the user's selection is data of the case where arrhythmia has been detected during the measurement (S202). Specifically, since the measurement data of the case where arrhythmia has been detected is stored in association with the fact that arrhythmia has been detected and the detail of the arrhythmia, it is determined whether or not the selected measurement data corresponds to such data. In a case where it is determined in step S202 that the measurement data according to the selection is not data of the case where arrhythmia has been detected during the measurement, the control unit 100 reads information on the measured blood pressure value (S203), displays the information on the display unit 12 (S205), and ends the series of processing.

On the other hand, in a case where it is determined in step S202 that the measurement data according to the selection is measurement data of the case where arrhythmia has been detected during the measurement, the control unit 100 reads the information on the arrhythmia (S204), displays a measurement result screen (screen as illustrated in FIG. 5A) indicating the fact that arrhythmia has been present without displaying the blood pressure value on the display unit 12 (S205), and ends the series of processing.

According to such processing, the user can view the past measurement result, and even in a case where arrhythmia has been detected at the time of the past measurement, it is possible to prevent an inaccurate blood pressure measurement result from being indicated to the user, and to notify the user of the arrhythmia detected during the blood pressure measurement.

### (Doctor-Side Terminal)

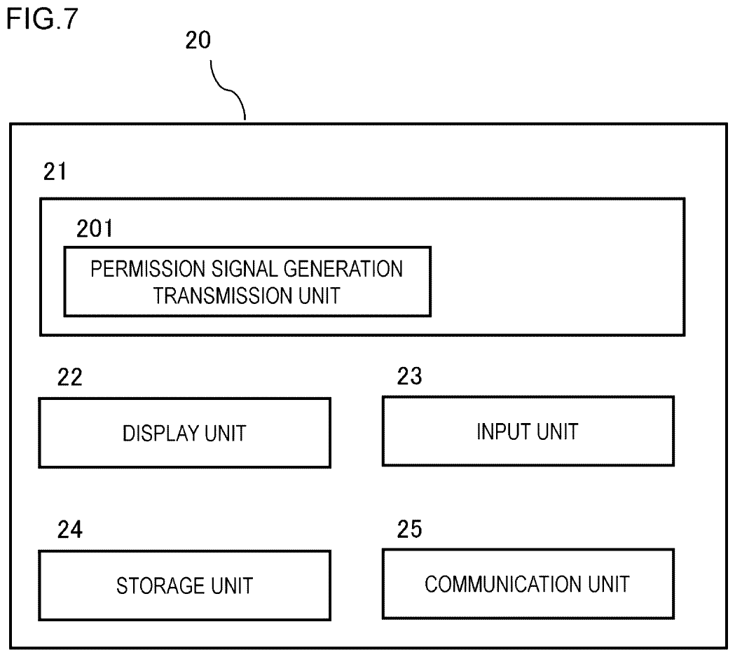

Next, the doctor terminal 20 will be described. FIG. 7 is a block diagram illustrating a functional configuration of the doctor terminal 20. The doctor terminal 20 is a general computer, for example, a stationarily installed personal computer, a portable notebook personal computer, a tablet terminal, or the like, and includes a control unit 21, a display unit 22, an input unit 23, a storage unit 24, and a communication unit 25.

The control unit 21 is a unit that controls the doctor terminal 20, and is configured by, for example, a CPU. The control unit 21 implements a functional module of a permission signal generation transmission unit 201. As will be described later, the permission signal generation transmission unit 201 generates a signal for permitting the display of the blood pressure value of the time when arrhythmia has been detected in the biological information measurement device 10, and transmits the signal to the biological information measurement device 10 via the communication unit 25.

Furthermore, the display unit 22 includes, for example, a display device such as a liquid crystal display. Furthermore, the input unit 23 is a unit that receives an operation input and an information input, for example, a keyboard, a mouse, a touch panel, a camera, a microphone, or the like. The storage unit 24 includes a main storage unit, an auxiliary storage unit, and the like, and stores an operating system (OS), various types of programs, and various types of data acquired via the communication network N. Furthermore, the communication unit 25 includes, for example, a communication interface board and a wireless communication circuit for wireless communication.

The doctor terminal 20 receives the measurement data from the biological information measurement device 10 via the communication network N, and outputs these pieces of information to the display unit 22. Although not illustrated, the doctor terminal 20 may be able to access the electronic medical record management system.

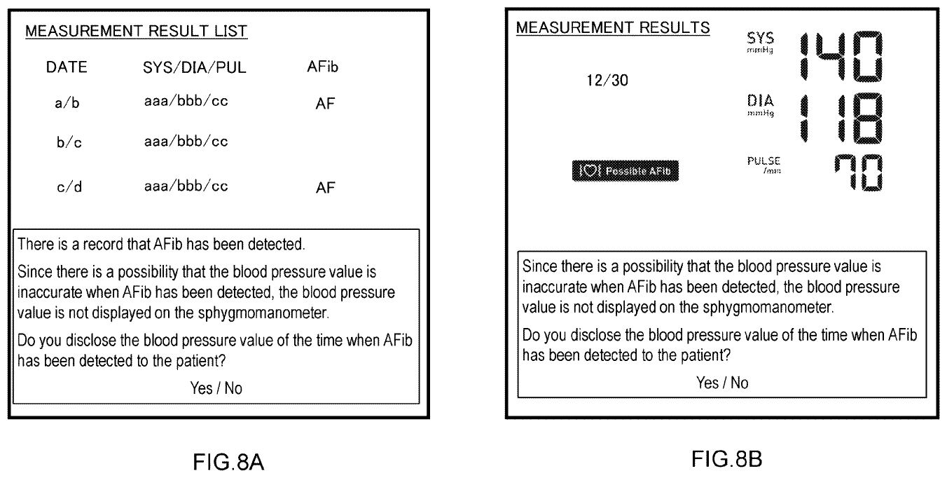

The doctor can refer to the measurement data transmitted from the biological information measurement device 10 and examine a medical care policy for the patient. FIGS. 8A and 8B are diagrams illustrating screen examples displayed on the display unit 22 of the doctor terminal 20. In the doctor terminal 20, a list display of the transmitted measurement data (see FIG. 8A) or a screen display showing individual measurement data (see FIG. 8B) can be provided on the display unit 22 by a user operation.

In any of the display screen examples of FIGS. 8A and 8B, the blood pressure value when arrhythmia has been detected during the measurement is displayed on the display unit 22 of the doctor terminal 20 together with the display indicating the fact that arrhythmia has been detected. In any of the display screen examples, in the biological information measurement device 10 used by the patient, there are displayed the fact that the blood pressure value of the time when arrhythmia has been detected is not displayed and a user interface to prompt the patient to input an operation whether or not to permit display of the blood pressure value of the time when arrhythmia has been detected.

Here, in a case where the doctor performs, via the input unit 23, an operation input for permitting the patient to display the blood pressure value of the time when the arrhythmia has been detected, the control unit 21 (permission signal generation transmission unit 201) of the doctor terminal 20 generates a signal (hereinafter, referred to as permission signal) for permitting the display of the blood pressure value of the case where arrhythmia has been detected during the measurement in the biological information measurement device 10, and transmits the signal via the communication unit 25.

### (Flows of Processing and Information in Information Processing System)

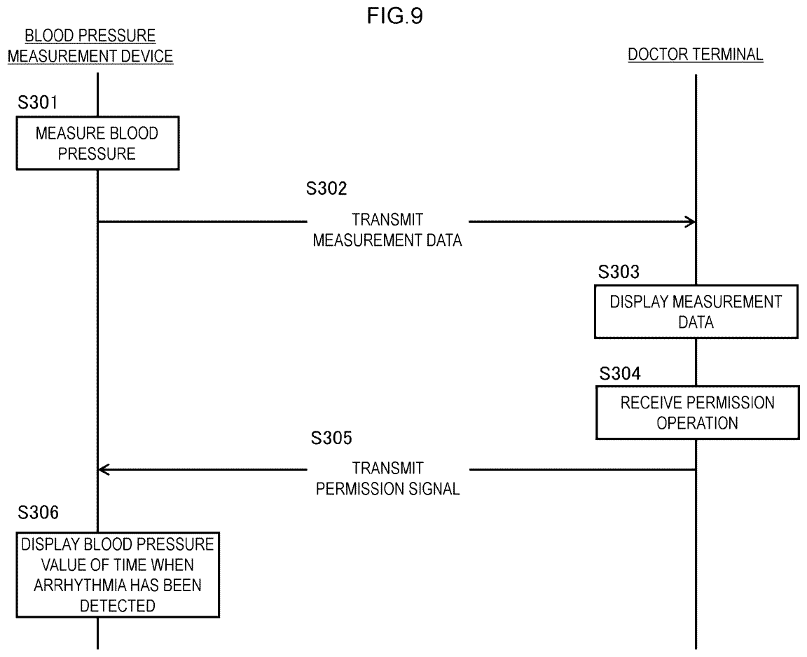

Next, a flow of processing performed in the information processing system 1 according to the present embodiment will be described on the basis of FIG. 9. FIG. 9 is a diagram illustrating a flow of processing performed in the information processing system 1 and transmission and reception of information. As illustrated in FIG. 9, first, the patient P performs blood pressure measurement with the biological information measurement device 10 (S301). Since the flow of the blood pressure measurement processing in the biological information measurement device 10 has already been described, the description thereof is omitted here. Then, the biological information measurement device 10 transmits the measurement data obtained in step S301 to the doctor terminal 20 (S302), and the measurement data is displayed on the doctor terminal 20 (display unit 22) (S303). Thereafter, in a case where the doctor terminal 20 receives an operation input for permitting the display of the blood pressure value of the time when the arrhythmia has been detected (S304), the doctor terminal 20 transmits a permission signal to the biological information measurement device 10 (S305). The biological information measurement device 10 that has received the permission signal releases the non-display state of the blood pressure value of the time when the arrhythmia has been detected, and thereafter, displays the fact that arrhythmia has been detected during the measurement and the calculated blood pressure value on the display unit 12 (S306).

According to the information processing system 1 according to the present embodiment as described above, it is possible to determine the presence or absence of arrhythmia during the blood pressure measurement, and in a case where arrhythmia has been detected, it is possible to prevent an inaccurate blood pressure measurement result from being indicated to the patient, and the doctor can confirm the blood pressure value of the time when the arrhythmia has been detected. Furthermore, after the doctor gives explanation or the like to the patient, the patient can confirm the measurement value of the time when the arrhythmia has been detected during the blood pressure measurement with the biological information measurement device.

### ((First Modification))

In the above embodiment, the biological information measurement device 10 is assumed to be a so-called sphygmomanometer that calculates a blood pressure value on the basis of a pressure pulse wave. However, as long as the biological information measurement device has a function for blood pressure measurement, the biological information measurement device may measure other biological information. Such a modification will be described below.

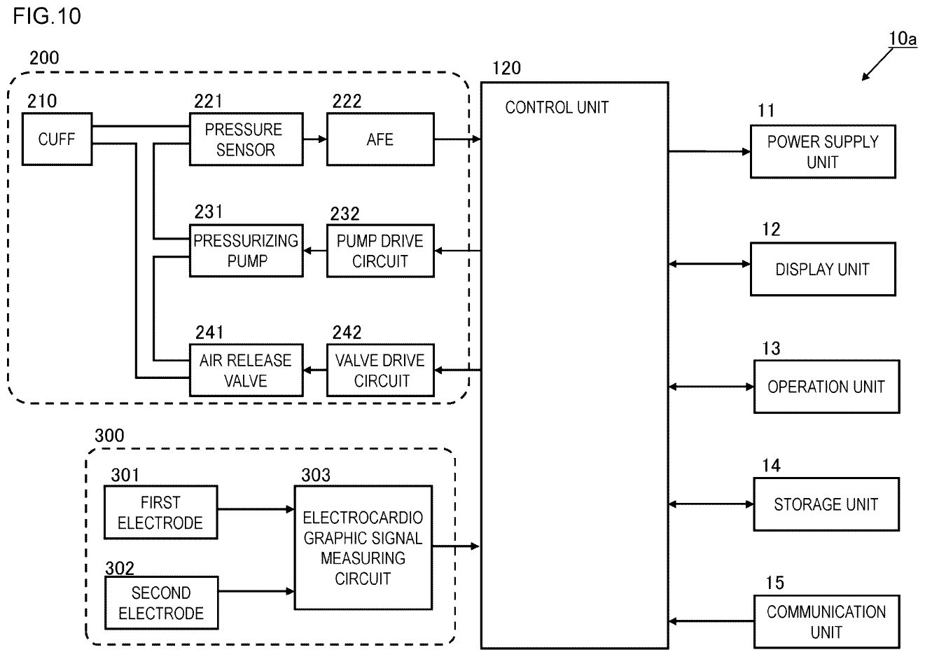

FIG. 10 is a block diagram illustrating a schematic configuration of a biological information measurement device 10a according to a first modification. The biological information measurement device 10a according to the present modification is a hybrid biological information measurement device capable of measuring not only blood pressure but also an electrocardiographic waveform. Note that, since the biological information measurement device 10a includes many configurations common to those of the biological information measurement device 10 of the first embodiment, hereinafter, the same reference numerals and signs are used for the same configurations as those of the first embodiment, and repeated description will be omitted.

As illustrated in FIG. 10, the biological information measurement device 10a includes an electrocardiographic waveform measurement unit 300 in addition to the same configurations as those of the biological information measurement device 10. The electrocardiographic waveform measurement unit 300 includes a plurality of electrodes (a first electrode 301 and a second electrode 302) and an electrocardiographic signal measuring circuit 303. The electrocardiographic signal measuring circuit 303 is a circuit for measuring an electrocardiographic waveform on the basis of an electrocardiographic signal obtained on the basis of a potential difference between two electrodes in contact with a living body, and includes an amplifier, a filter, an A/D conversion circuit, and the like.

Although not illustrated, the first electrode 301 and the second electrode 302 are disposed at positions separated from each other in the main body housing. When the user performs measurement processing in a state where one of the left and right fingers is in contact with the first electrode 301 and the other is in contact with the second electrode 302, the potential difference between the first electrode 301 and the second electrode 302 is measured as an electrocardiographic waveform by the electrocardiographic signal measuring circuit 303 (so-called I induction), and the electrocardiographic waveform (electrocardiogram) is measured by recording the potential difference.

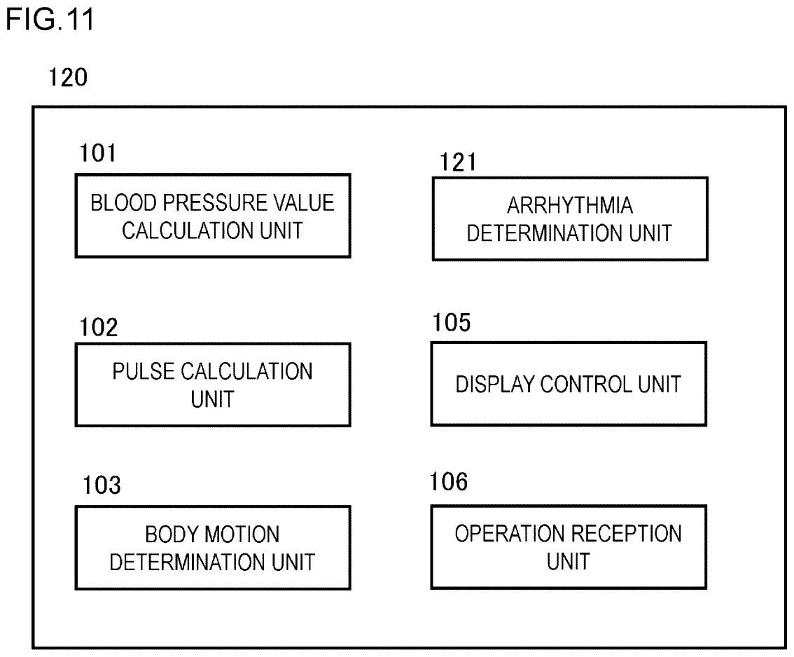

Furthermore, as illustrated in FIG. 11, the biological information measurement device 10a includes functional modules including the blood pressure value calculation unit 101, the pulse calculation unit 102, the body motion determination unit 103, the display control unit 105, and an arrhythmia determination unit 121 which are implemented by a control unit 120.

In the present modification, the arrhythmia determination unit 121 detects arrhythmia on the basis of not the pressure pulse wave but the electrocardiographic waveform measured by the electrocardiographic signal measurement circuit 303. Specifically, by simultaneously performing the blood pressure measurement by the blood pressure measurement unit 200 and the electrocardiographic waveform measurement by the electrocardiographic waveform measurement unit 300, the arrhythmia determination unit 121 determines the presence or absence of arrhythmia during the blood pressure measurement on the basis of the electrocardiographic waveform acquired during the blood pressure measurement. That is, in the present embodiment, a combination of the arrhythmia determination unit 121 and the electrocardiographic waveform measurement unit 300 corresponds to the arrhythmia information acquisition unit.

Note that the flow of processing of the blood pressure measurement in the biological information measurement device 10a according to the present modification is similar to that of the biological information measurement device 10 of the first embodiment except that the detection of arrhythmia is performed on the basis of the electrocardiographic waveform instead of the pressure pulse wave, and the display contents of the blood pressure measurement result, the viewing of the past history data of the blood pressure values, and the like are also similar to those of the biological information measurement device 10.

As in the biological information measurement device 10a according to the present modification, by detecting arrhythmia on the basis of the electrocardiographic waveform, it is possible to more accurately detect arrhythmia during the blood pressure measurement.

### ((Second Modification))

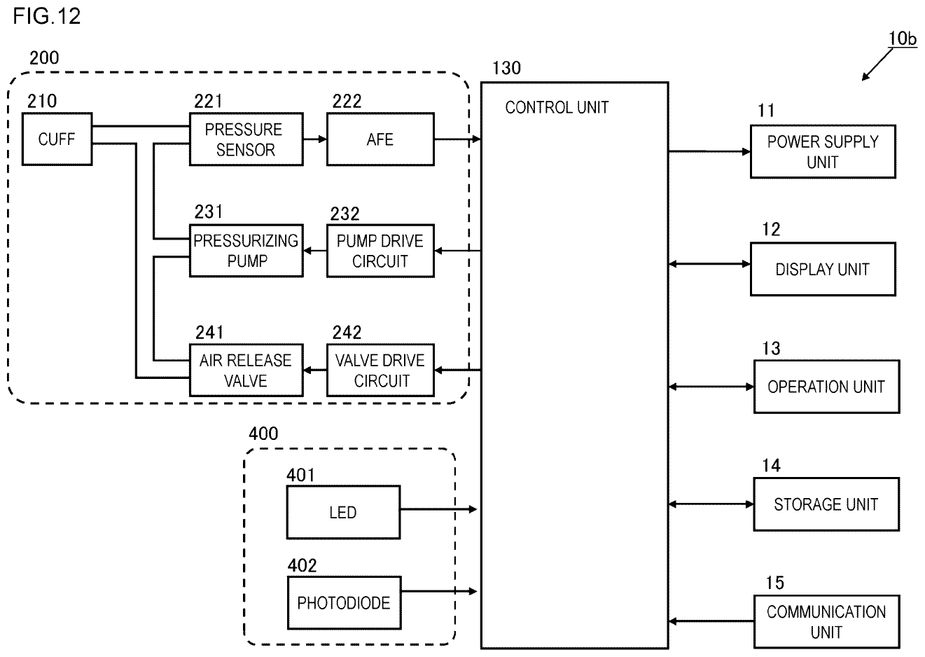

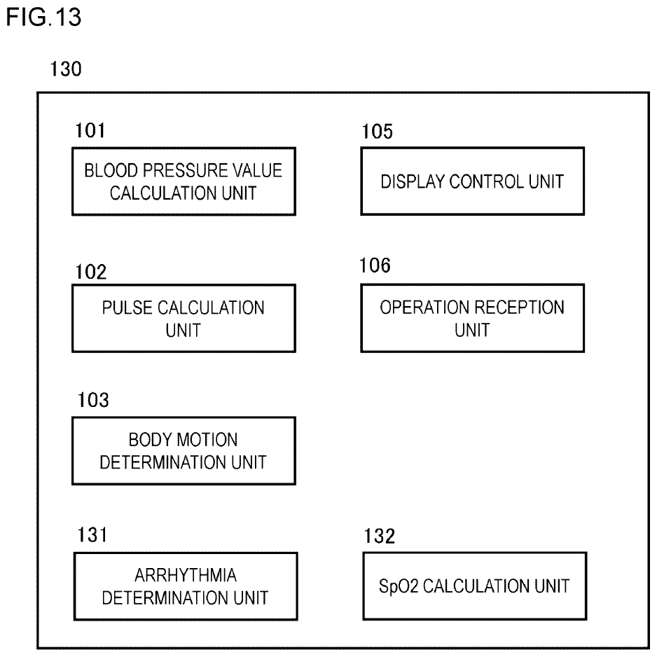

Subsequently, another modification will be described on the basis of FIGS. 12 and 13. A biological information measurement device 10b according to the present modification is a hybrid biological information measurement device capable of measuring not only blood pressure but also blood oxygen saturation (SpO2).

As illustrated in FIG. 12, the biological information measurement device 10b includes an SpO2 measurement unit 400 in addition to the same configurations as those of the biological information measurement device 10. The SpO2 measurement unit 400 includes an LED 401 as a light emitting element and a photodiode 402 as a light receiving element, and acquires information for calculating blood oxygen saturation by receiving reflected light of red light or infrared light emitted from the LED 401 by the photodiode 402.

Note that the SpO2 measurement unit 400 can not only measure the blood oxygen saturation but also detect a change in the blood flow rate accompanying the heartbeat as a change in the reflected light received by the photodiode 402 and acquire the pulse wave on the basis of the change.

Furthermore, as illustrated in FIG. 13, the biological information measurement device 10b includes functional modules including the blood pressure value calculation unit 101, the pulse calculation unit 102, the body motion determination unit 103, the display control unit 105, an arrhythmia determination unit 131, and an SpO2 calculation unit 132 which are implemented by the control unit 130.

The SpO2 calculation unit 132 calculates the blood oxygen concentration using the information acquired by the SpO2 measurement unit 400 (the intensity of the reflected light received by the photodiode 402).

Furthermore, in the present modification, the arrhythmia determination unit 131 detects arrhythmia on the basis of not the pressure pulse wave but the photoelectric pulse wave acquired by the SpO2 measurement unit 400. Specifically, by simultaneously performing the blood pressure measurement by the blood pressure measurement unit 200 and the measurement by the SpO2 measurement unit 400, the arrhythmia determination unit 131 determines the presence or absence of arrhythmia during the blood pressure measurement on the basis of the pulse wave information acquired by the SpO2 measurement unit 400. That is, in the present embodiment, a combination of the arrhythmia determination unit 131 and the SpO2 measurement unit 400 corresponds to the arrhythmia information acquisition unit.

Note that the flow of processing of the blood pressure measurement in the biological information measurement device 10b according to the present modification is similar to that of the biological information measurement device 10 of the first embodiment except that the detection of arrhythmia is performed on the basis of the photoelectric pulse wave instead of the pressure pulse wave, and the display contents of the blood pressure measurement result, the viewing of the past history data of the blood pressure values, and the like are also similar to those of the biological information measurement device 10.

### Second Embodiment

### (System Configuration)

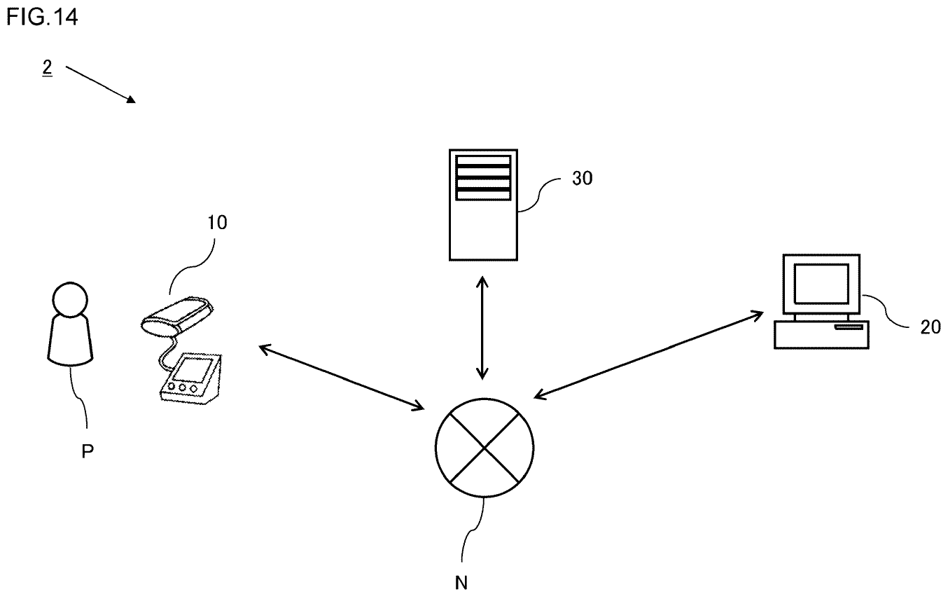

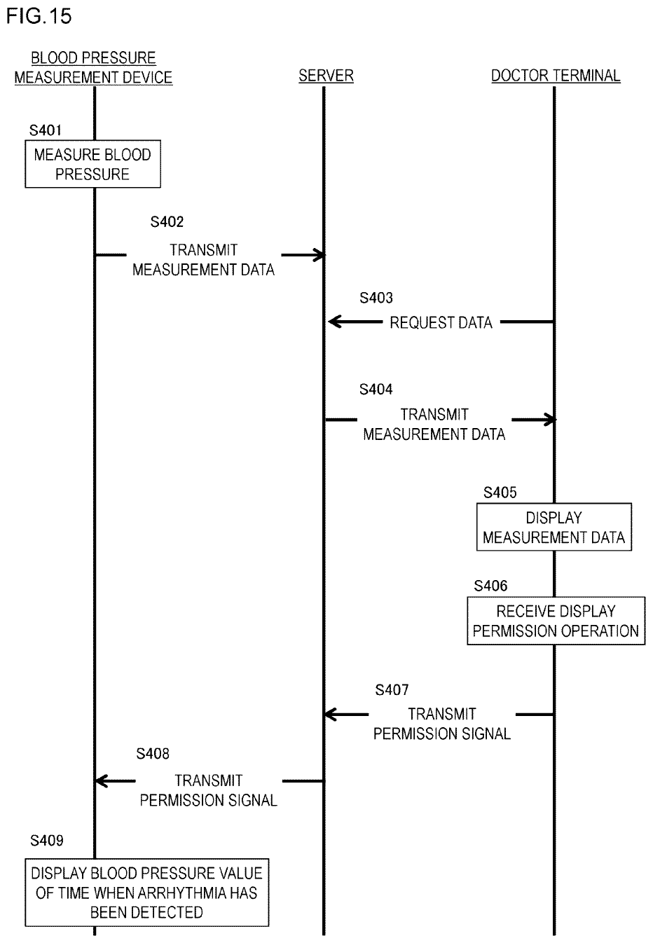

Next, still another embodiment of the present invention will be described on the basis of FIGS. 14 and 15. As illustrated in FIG. 14, an information processing system 2 according to the present embodiment includes a server device 30 connected to the network N in addition to the information processing system 1 of the first embodiment. Note that the biological information measurement device 10 and the doctor terminal 20 are similar to those of the first embodiment. The server device 30 is configured by a general server computer, and includes a control unit, a communication unit, a storage unit, and the like (none of which are illustrated).

In the present embodiment, the biological information measurement device 10 and the doctor terminal 20 do not directly transmit and receive information, and the measurement data is stored in the server device 30, and the server device 30 transmits or allows viewing of the measurement data in response to a request from the doctor terminal 20. Furthermore, a permission signal in a case where the doctor permits display of the blood pressure value measured at the time when arrhythmia has been detected is also transmitted to the biological information measurement device 10 via the server device 30. The permission signal may be generated in the doctor terminal 20 and stored in the server device, or may be generated by the server device 30 on the basis of a command from the doctor terminal 20.

### (Flow of Processing)

Subsequently, a flow of processing performed in the information processing system 2 according to the present embodiment and transmission and reception of information will be described on the basis of FIG. 15. As illustrated in FIG. 15, first, the patient P performs blood pressure measurement with the biological information measurement device 10 (S401). Since the flow of the blood pressure measurement processing in the biological information measurement device 10 has already been described, the description thereof is omitted here. Then, the biological information measurement device 10 transmits the measurement data obtained in step S401 to the server device 30 (S402), and the measurement data is stored in the server device 30.

Thereafter, when a transmission request of the measurement data (not only individual data but also a plurality of pieces of data for a predetermined period) is made from the doctor terminal 20 (S403), the server device 30 transmits the measurement data related to the request to the doctor terminal 20 (S404). Then, the requested measurement data is displayed on the doctor terminal 20 (S405). Thereafter, in a case where the doctor terminal 20 receives an operation input for permitting the display of the blood pressure value of the time when the arrhythmia has been detected (S406), the doctor terminal 20 generates a permission signal and transmits the permission signal to the server device 30 (S407).

The server device 30 that has received the permission signal stores the permission signal in the storage unit, and transmits the permission signal to the biological information measurement device 10 (S408). Then, the biological information measurement device 10 that has received the permission signal releases the non-display state of the blood pressure value of the time when the arrhythmia has been detected, and thereafter, displays the fact that arrhythmia has been detected during the measurement and the calculated blood pressure value on the display unit 12 (S409).

According to the information processing system 2 having the above configuration, it is not necessary to store a large amount of measurement data in the doctor terminal 20, and the cost for securing the storage capacity can be reduced. Furthermore, also from the viewpoint of information management, it is possible to reduce the burden and risk on the doctor and the medical institution using the doctor terminal 20.

In the present embodiment, an example in which the measurement data and the permission signal are stored in the server device 30 and transmitted to the doctor terminal 20 and the biological information measurement device 10 (that is, an example in which the data can be downloaded) has been described. However, functions such as viewing the measurement data and receiving permission to display the blood pressure value of the time when arrhythmia has been detected may be provided without downloading the data from the server device 30.

### Third Embodiment

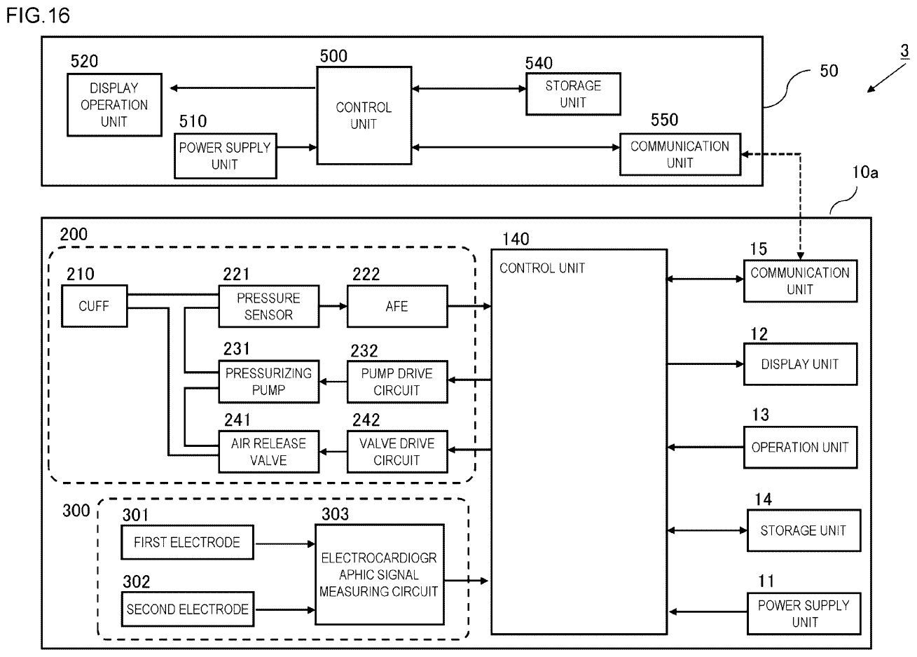

In the above embodiment, the biological information measurement device 10 is configured to be connected to the doctor terminal 20 and the server device 30 at a long distance via the network N. However, the biological information measurement device 10 and a specific terminal may be connected by short distance communication (by pairing or the like). Hereinafter, such an information processing system 3 will be described with reference to FIGS. 16 to 20.

FIG. 16 is a block diagram illustrating a schematic configuration of the information processing system 3 according to the present embodiment. As illustrated in FIG. 16, the information processing system 3 according to the present embodiment includes the biological information measurement device 10a including the blood pressure measurement unit 200 and the electrocardiographic waveform measurement unit 300, and a smartphone 50 as an example of the information processing terminal, which are configured to be communicably connectable. Note that a communication method between the biological information measurement device 10a and the smartphone 50 is not particularly limited, and for example, pairing by Bluetooth (registered trademark) low energy (BLE) communication, LAN communication by Wi-Fi (registered trademark), infrared communication, or the like can be adopted.

As illustrated in FIG. 16, the smartphone 50 includes configurations such as a power supply unit 510, a display operation unit 520, a storage unit 540, a communication unit 550, and a control unit 500. Note that, since the biological information measurement device 10a has the same configuration as that described in the first modification of the first embodiment, the description thereof will not be repeated.

The power supply unit 510 is a power source of the smartphone 50, and can be, for example, a rechargeable secondary battery such as a lithium ion battery. The display operation unit 520 is, for example, a touch panel display or the like, and has both functions of information display and input operation reception.

The storage unit 540 includes a main storage device such as a RAM and a ROM, and an auxiliary storage device such as a flash memory, and stores various types of information such as an application program and various pieces of biological information. Note that the measurement results of the blood pressure and the like and other acquired biological information may be stored in the storage unit 540 in association with time information such as the measurement (acquisition) time. As the time information, for example, time information clocked by referring to an RTC of the control unit 500 can be used. Furthermore, the auxiliary storage device may be configured to be detachable from the main body housing (not illustrated).

The communication unit 550 functions to communicate with and connect to the biological information measurement device 10a, and can adopt an appropriate communication interface such as a wired communication port or a wireless communication antenna.

The control unit 500 is a functional unit that controls the smartphone, and includes, for example, a processor such as a CPU and a GPU. When receiving a user's operation via the display operation unit 520, the control unit 500 controls each component of the smartphone to exhibit various functions according to a predetermined application program. Note that the predetermined program is stored in the storage unit 540 and read therefrom.

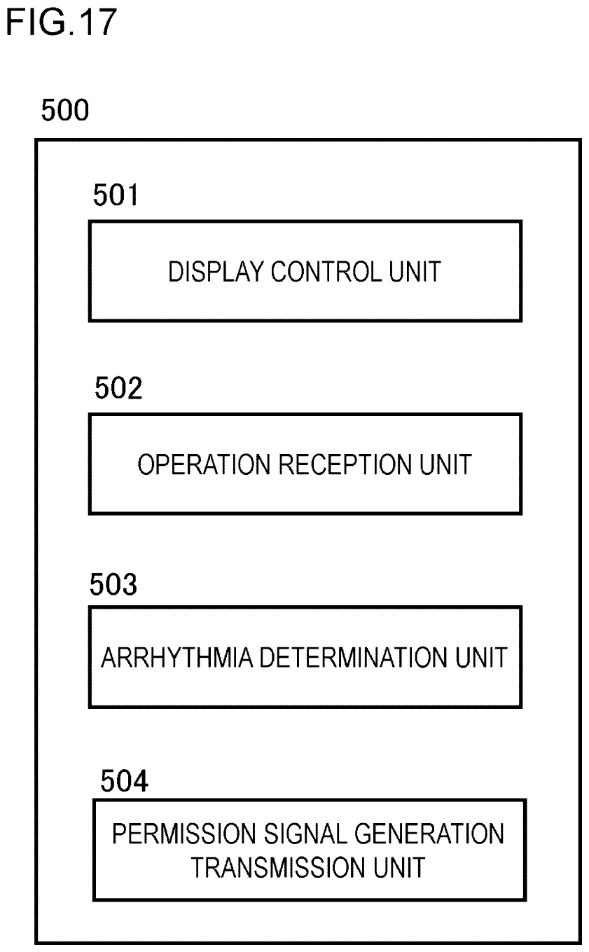

FIG. 17 is a block diagram illustrating part of the functional modules implemented by the control unit 500 of the smartphone 50. As illustrated in FIG. 17, the control unit 500 of the smartphone 50 implements functional modules including a display control unit 501, an operation reception unit 502, an arrhythmia determination unit 503, and a permission signal generation transmission unit 504.

The display control unit 501 controls the display contents of the display operation unit 520. For example, the display control unit 501 causes the display operation unit 520 to display a menu screen (operation reception screen), guidance related to the progress of biological information measurement, a measurement result of biological information, and the like according to an application program related to cooperation with the biological information measurement device 10a.

The operation reception unit 502 receives an input of a user operation via a user interface displayed on the display operation unit 520. For example, in accordance with an application program related to cooperation with the biological information measurement device 10a, the operation reception unit 502 receives an operation input such as start of measurement, selection of an item, and screen transition by the biological information measurement device 10a.

As will be described later, the arrhythmia determination unit 503 determines (detects), on the basis of the information on the electrocardiographic waveform transmitted from the biological information measurement device 10a, the presence or absence of arrhythmia at the time of blood pressure measurement performed simultaneously with the acquisition of the electrocardiographic waveform. That is, in the present embodiment, the detection processing of arrhythmia during the blood pressure measurement can be performed by the smartphone 50. Therefore, the biological information measurement device 10a does not need to include the functional modules of the arrhythmia determination unit 121.

As will be described later, the permission signal generation transmission unit 504 generates a signal for permitting the display of the blood pressure value of the time when arrhythmia has been detected, and transmits the permission signal to the biological information measurement device 10a via the communication unit 550.

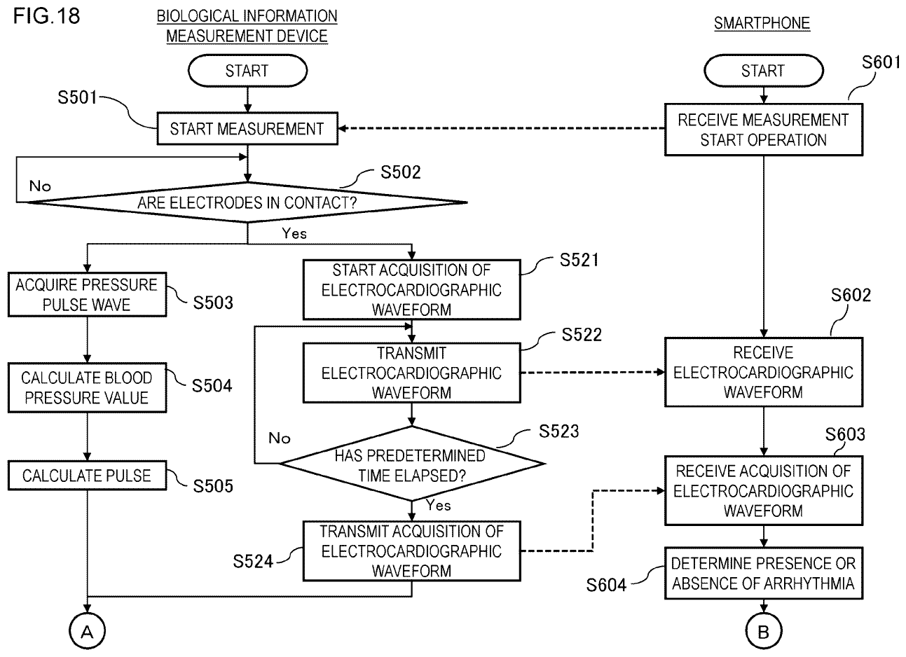

Next, a flow of processing performed in the information processing system 3 according to the present embodiment will be described on the basis of FIGS. 18 to 20. FIG. 18 is a diagram illustrating a flow of processing performed in the information processing system 3 and transmission and reception of information. In the information processing system 3 according to the present embodiment, the smartphone 50 can instruct to start the collective measurement of the blood pressure and the electrocardiographic waveform. Therefore, the control unit 500 (operation reception unit 502) of the smartphone 50 receives the operation input of starting the collective measurement of the blood pressure and the electrocardiogram via the display operation unit 520 (S601). The instruction by the operation input is transmitted from the smartphone 50 to the biological information measurement device 10a via the communication unit 550, and the biological information measurement device 10a starts the collective measurement of the blood pressure and the electrocardiogram (S501).

When the collective measurement is started, the biological information measurement device 10a determines whether or not the first electrode 301 and the second electrode 302 are correctly in contact with the living body on the basis of the signal acquired by the electrocardiographic waveform measurement unit 300 (S502). Here, in a case where it is determined that both electrodes are not correctly in contact with the living body, the processing to determine the electrode contact is repeatedly performed. On the other hand, in a case where it is determined in step S502 that both electrodes are correctly in contact with the living body, the biological information measurement device 10a executes acquisition of the pressure pulse wave by the blood pressure measurement unit 200 and acquisition of the electrocardiographic waveform by the electrocardiographic waveform measurement unit 300 in parallel (S503, S521).

The electrocardiographic waveform acquired in step S521 by the biological information measurement device 10a is transmitted to the smartphone 50 via the communication unit 15 (S522), and the smartphone 50 receives the electrocardiographic waveform (S602). The biological information measurement device 10a (control unit 120) continues to acquire the electrocardiographic waveform, and determines whether or not a predetermined time (for example, time sufficient for pulse wave acquisition for blood pressure measurement) has elapsed (S523). Here, in a case where it is determined that the predetermined time has not elapsed, the process returns to step S522, and the subsequent processing is repeated.

On the other hand, in a case where it is determined in step S523 that the predetermined time has elapsed, the biological information measurement device 10a ends the acquisition of the electrocardiographic waveform and transmits that fact to the smartphone 50 via the communication unit 15 (S524), and the smartphone 50 receives the information of the fact that the acquisition of the electrocardiographic waveform has ended (S603). Then, in the smartphone 50 that has received the electrocardiographic waveform for the predetermined time, the control unit 500 (arrhythmia determination unit 503) executes analysis processing of determining (detecting) the presence or absence of arrhythmia during the blood pressure measurement (during the acquisition of the pressure pulse wave) on the basis of the electrocardiographic waveform (S604).

On the other hand, in the biological information measurement device 10a, in parallel with the acquisition of the electrocardiographic waveform, acquisition of a pressure pulse wave for blood pressure measurement is performed by the blood pressure measurement unit 200 (S503). In addition, based on the acquired pressure pulse wave, the control unit 120 (the blood pressure value calculation unit 101 and the pulse calculation unit 102) calculates the blood pressure value (S504), and calculates the pulse rate (S505).

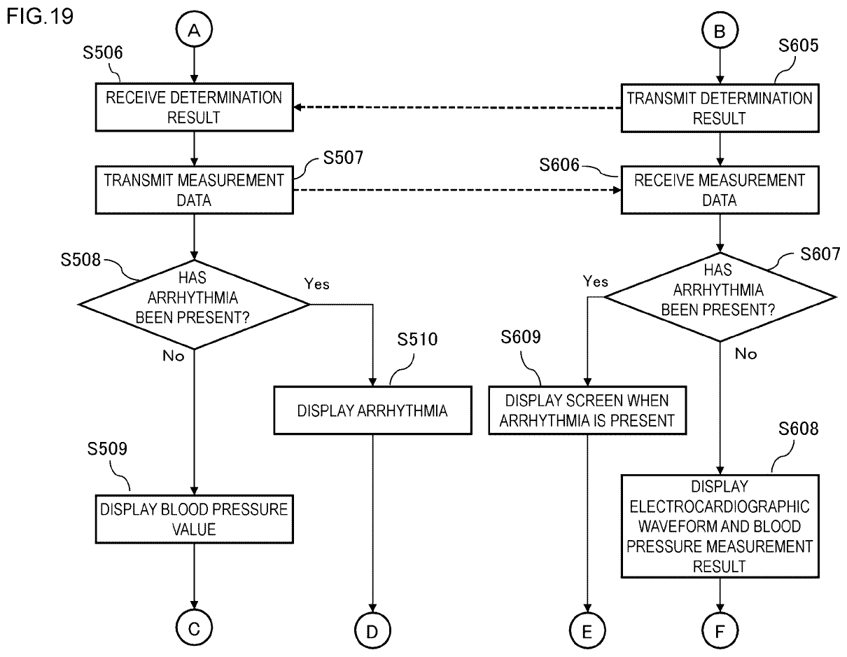

Subsequently, referring to FIG. 19, the smartphone 50 transmits the determination result of the presence or absence of arrhythmia performed in step S604 to the biological information measurement device 10a via the communication unit 550 (S605), and the biological information measurement device 10a receives the determination result (S506). Furthermore, the biological information measurement device 10a transmits the calculated blood pressure value and pulse rate (hereinafter, collectively referred to as blood pressure measurement data) to the smartphone 50 via the communication unit 15 (S507), and the smartphone 50 receives the blood pressure measurement data (S606).

In a case where the determination result received in step S506 indicates that arrhythmia has been present during the blood pressure measurement, the biological information measurement device 10a (display control unit 105) does not display the calculated blood pressure value but displays, as a measurement result, the fact that arrhythmia has been present on the display unit 12 (S508, S510).

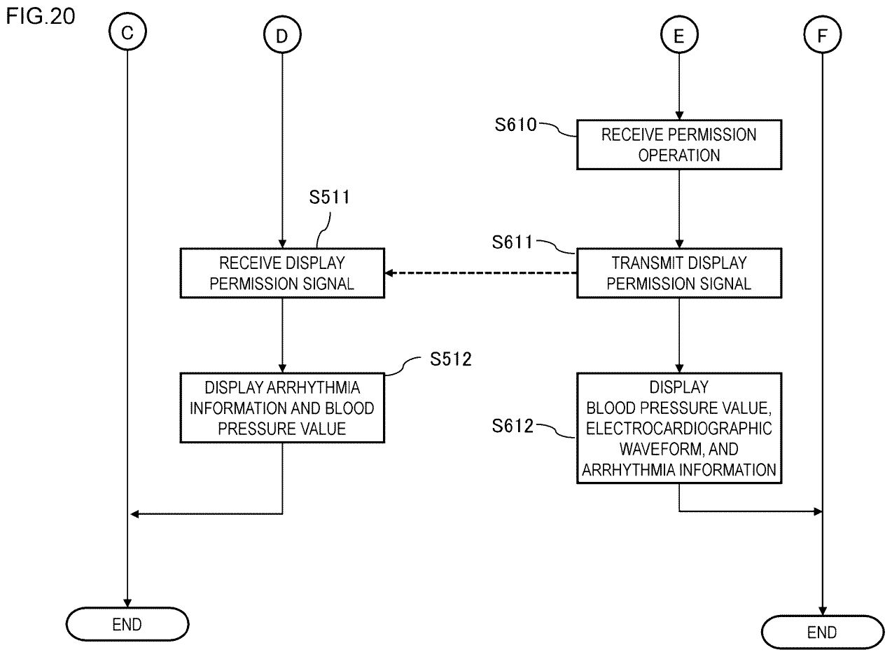

Then, in a case where the determination result received in step S506 indicates that arrhythmia has been absent during the blood pressure measurement, the biological information measurement device 10a (display control unit 105) displays the blood pressure value and the pulse rate on the display unit 12 (S508, S509), and ends the series of processing (see FIG. 20).

In a case where the result of step S605 is the presence of the arrhythmia, the smartphone 50 suspends the display of the blood pressure measurement data and displays, on the display operation unit 520, a screen including a message indicating that arrhythmia has been detected during the blood pressure measurement and that the measured blood pressure value is not accurate, and an interface whether or not to display the measured blood pressure value knowing that the measured blood pressure value is an inaccurate value (S607, S609).

On the other hand, in a case where the result of step S605 is absence of the arrhythmia, the smartphone 50 displays the blood pressure measurement data and the electrocardiographic waveform (electrocardiogram) on the display operation unit 520 (S607, S608), and ends the series of processing (see FIG. 20).

Next, referring further to FIG. 20, after step S609, the smartphone 50 receives an operation input by the user that permits the display of the measurement value of the blood pressure of the time when the arrhythmia has been detected (S610). After receiving the permission operation by the user, the smartphone 50 (permission signal generation transmission unit 504) generates a permission signal for displaying the blood pressure value of the time when the arrhythmia has been detected in the biological information measurement device 10a, and transmits the permission signal to the biological information measurement device 10a via the communication unit 550 (S611). Then, when the transmitted display permission signal is received in the biological information measurement device 10a (S511), the biological information measurement device 10a (display control unit 105) displays, on the display unit 12, the measured blood pressure value and the object notifying that arrhythmia has occurred during the blood pressure measurement (S512), and ends the series of processing.

On the other hand, also in the smartphone 50, in a case where the display permission operation is received in step S610, the blood pressure measurement data including the blood pressure value whose display has been suspended, the electrocardiographic waveform (electrocardiogram), and the object notifying that arrhythmia has occurred during the blood pressure measurement are displayed on the display operation unit 520 (S612), and the series of processing is ended.

Note that the order of the processing described above can be appropriately changed or omitted, and for example, it is not clear which of the blood pressure value calculation and the arrhythmia presence/absence determination ends first, so that the order of step S506 and step S507 (and step S605 and step S606) may be reversed. Alternatively, step S506 (reception of determination result of presence or absence of arrhythmia) may be performed before step S504 (calculation of blood pressure value in the biological information measurement device 10a).

In the above example, in step S523, the biological information measurement device 10a determines whether or not the predetermined time has elapsed from the start of acquisition of the electrocardiographic waveform. However, this processing may be executed on the smartphone 50 side. In this case, the smartphone 50 may transmit a command to end the acquisition of the electrocardiographic waveform to the biological information measurement device 10a after the predetermined time elapses.

In the smartphone 50, after the electrocardiographic waveform is received in step S602, the electrocardiographic waveform may be displayed on the display operation unit 520 in real time. Furthermore, the smartphone 50 may not display the blood pressure measurement data and the electrocardiographic waveform regardless of the presence or absence of the arrhythmia, and may perform only the display permission operation and the generation and transmission of the display permission signal to the biological information measurement device 10a.

According to the information processing system 3 as described above, the determination of the presence or absence of arrhythmia during the blood pressure measurement can be executed by the information processing terminal such as the smartphone 50 instead of the biological information measurement device 10a. As a result, the calculation load of the biological information measurement device 10a can be reduced, and the present invention can also be used for the biological information measurement device 10a including a non-high-performance processor.

### ((Modification))

However, the smartphone 50 does not necessarily need to determine the presence or absence of arrhythmia during the blood pressure measurement, and the biological information measurement device 10a may be configured to determine the presence or absence of arrhythmia and transmit the result to the smartphone 50. That is, the smartphone 50 may perform only reception of the display permission operation for permitting display of the blood pressure value of the time when arrhythmia has been detected during the measurement in the biological information measurement device 10a and generation and transmission of the display permission signal.

### Others

Note that the description of the above-described embodiments is merely illustrative of the present invention, and the present invention is not limited to the above-described specific forms. The present invention can be variously modified and combined within the scope of the technical idea. For example, the configuration according to the second embodiment and the configuration according to the third embodiment may be combined to form a biological information measurement device including all of the blood pressure measurement unit 200, the electrocardiographic waveform measurement unit 300, and the SpO2 measurement unit 400.

In the above embodiments, when the fact that arrhythmia has been detected during the blood pressure measurement is displayed, the display mode may be made different from the display mode at the time of notifying of another measurement error or failure of the device. Specifically, the detection of arrhythmia can be clearly identified from other errors by a difference in lighting/blinking of the display, a difference in blinking interval, a difference in display color, and the like.

In the above description, the electrocardiographic waveform measurement unit 300 and the SpO2 measurement unit 400 are exemplified as the devices integrated with the blood pressure measurement unit 200. However, the present invention is not limited thereto, and a configuration can be adopted in which the electrocardiographic waveform and the photoelectric pulse wave acquired by a separate measuring instrument are acquired via the communication unit. In this case, the communication unit serves as the arrhythmia information acquisition unit. Furthermore, the present invention can also be applied to a wearable type, such as a wristwatch type, biological information measurement device.

In the third embodiment, the biological information measurement device 10a includes the blood pressure measurement unit 200 and the electrocardiographic waveform measurement unit 300. However, determining the presence or absence of arrhythmia during the blood pressure measurement using another information processing terminal is naturally applicable to other biological information measurement devices as described in the first embodiment. Furthermore, the information processing terminal is not limited to the smartphone 50, and other computers can be adopted regardless of whether the computer is a portable computer or a stationary computer.

In each of the above examples, the description has been made using the atrial fibrillation as an example of arrhythmia. However, as a matter of course, also in a case where another arrhythmia (atrial flutter, premature contraction, etc.) has been detected, the fact that atrial flutter or premature contraction has been detected may be displayed without displaying the blood pressure value similarly to the above embodiments.

What is claimed is:

1. A biological information measurement device comprising:
a blood pressure measurement unit that acquires information for measuring blood pressure of a living body;
an arrhythmia information acquisition unit that acquires information related to presence or absence of arrhythmia in the living body during measurement of the blood pressure;
a display unit including an area capable of displaying at least a blood pressure value measured;
a storage unit that stores at least the blood pressure value;
a communication unit for communicating with another device; and
a control unit, wherein
the control unit
calculates the blood pressure on the basis of the information acquired by the blood pressure measurement unit,
causes, in a case where the information acquired by the arrhythmia information acquisition unit indicates that arrhythmia during the measurement of the blood pressure has been present, the display unit not to display a value of the blood pressure measured but to display information of a fact that arrhythmia has been present, and
causes, after causing the information of the fact that arrhythmia has been present to be displayed, in a case where a permission signal to permit display of a blood pressure value measured when the arrhythmia has been present is received via the communication unit, the display unit to display the blood pressure value.

2. The biological information measurement device according to claim 1, wherein
the arrhythmia information acquisition unit acquires the information related to presence or absence of arrhythmia in the living body on the basis of pulse wave information acquired by the blood pressure measurement unit.

3. The biological information measurement device according to claim 1, further comprising
an electrocardiographic waveform acquisition unit that acquires an electrocardiographic waveform of the living body, wherein
the arrhythmia information acquisition unit acquires the information related to presence or absence of arrhythmia in the living body on the basis of the electrocardiographic waveform during the measurement of the blood pressure.

4. An information processing system comprising:
the biological information measurement device according to claim 1; and an information processing terminal communicatively connected to the biological information measurement device, wherein
the biological information measurement device
transmits, to the information processing terminal, information at least related to presence or absence of arrhythmia during the measurement of the blood pressure, and wherein
the information processing terminal includes:
a terminal-side display unit that displays, in a case where the information of the fact that arrhythmia has been present during the measurement of the blood pressure is received, at least the fact; and
an input unit that receives a display permission operation that is an operation for permitting display of the blood pressure value measured when the arrhythmia has been present, and
transmits, in a case where the display permission operation is received, the permission signal to the biological information measurement device.

5. The information processing system according to claim 4, wherein
the biological information measurement device
transmits information on the blood pressure value measured to the information processing terminal, and
the information processing terminal
displays the information on the blood pressure value received on the terminal-side display unit, and displays, in a case where the blood pressure value to be displayed is a blood pressure value measured when the arrhythmia has been detected, a fact that the blood pressure value displayed is a blood pressure value measured when arrhythmia has been detected together with the blood pressure value.

6. The information processing system according to claim 5, wherein
the biological information measurement device
transmits the information on the blood pressure value measured to the information processing terminal, and
the information processing terminal
suspends, in a case where the blood pressure value received is a blood pressure value measured when the arrhythmia has been detected, display of the blood pressure value on the terminal-side display unit and displays a fact that the blood pressure value received is the blood pressure value of a case where arrhythmia has been detected during the measurement on the terminal-side display unit, and displays, only in a case where the display permission operation is received, the blood pressure value received on the terminal-side display unit.

7. An information processing system comprising:
a biological information measurement device configured to be able to measure at least blood pressure of a living body; and an information processing terminal communicatively connected to the biological information measurement device, wherein
the biological information measurement device
acquires information for measuring the blood pressure of the living body and calculates a blood pressure value,
acquires determination information for determining presence or absence of arrhythmia in the living body during measurement of the blood pressure and transmits the determination information to the information processing terminal,
suspends, in a case where a signal of a fact that arrhythmia during the measurement of the blood pressure has been present is received from the information processing terminal, display of a value of the blood pressure measured and displays information of the fact that arrhythmia has been present, and
displays, after displaying the information of the fact that arrhythmia has been present, in a case where a permission signal to permit display of a blood pressure value measured when the arrhythmia has been present is received from the information processing terminal, the blood pressure value, and wherein
the information processing terminal
receives the determination information from the biological information measurement device,
determines presence or absence of arrhythmia during measurement of the blood pressure of the living body on the basis of the determination information,
transmits, in a case where it is determined that arrhythmia has been present during the measurement of the blood pressure of the living body, information of a fact that arrhythmia has been present during the measurement of the blood pressure of the living body to the biological information measurement device and receives a display permission operation that is an operation for permitting display of a blood pressure value measured when the arrhythmia has been present, and
transmits, in a case where the display permission operation is received, the permission signal to the biological information measurement device.

8. The information processing system according to claim 7, wherein
the biological information measurement device includes an electrocardiographic waveform acquisition unit that acquires an electrocardiographic waveform of the living body, and
the determination information is the electrocardiographic waveform.

9. A non-transitory computer-readable storage medium recording a program for causing an information processing device to function as the information processing terminal according to claim 7.
